(19) United States
(12) Patent Application Publication
(10) Pub. No.: US 2016/0270930 A1
(43) Pub. Date: Sep. 22, 2016

(54) SYSTEM AND METHOD FOR DEPLOYING IMPLANT

(71) Applicant: NLT SPINE LTD., Kfar Saba (IL)

(72) Inventors: Tzony SIEGAL, Moshav Shoeva (IL); Oded LOEBL, Tel Mond (IL); Didier TOUBIA, Raanana (IL)

(73) Assignee: NLT Spine Ltd., Kfar Saba (IL)

(21) Appl. No.: 14/442,405

(22) PCT Filed: Dec. 4, 2013
(86) PCT No.: PCT/IB2013/060639

§ 371 Date: May 13, 2015

Related U.S. Application Data
(60) Provisional application No. 61/732,955, filed on Dec. 4, 2012.

(51) Int. Cl. A61F 2/46; A61F 2/44

(52) U.S. Cl. CPC A61F 2/4611; A61F 2/447; A61F 2002/30471

(57) ABSTRACT

A system and method for deploying a medical implant includes a conduit assembly having a second conduit portion rigidly attached to a distal end of a first conduit portion such that inner channels of the two conduit portions are contiguous. The second conduit portion provides first and second opposing tissue contact surfaces rigidly interconnected by a rigid bridging structure. An implant insert is deployed along the inner channel of the first conduit portion to a deployed position engaging the second conduit portion. The second conduit portion is selectively detachable from the first conduit portion so as to leave the second conduit portion and the implant insert together defining an implant.

### FIELD AND BACKGROUND OF THE INVENTION

The present invention relates to medical devices and, in particular, it concerns a system and method for deploying an implant within a body.

Implants are used in a wide range of surgical procedures. Particularly in the field of orthopedic implants, an implant may need to be inserted between two regions of tissue after distraction to open up a sufficient space to receive the implant.

In various procedures, use of a delivery conduit has been found useful. A conduit is typically an elongated rigid structure which is robust, relatively easy to manipulate, can be helpful in defining the target region for performance of a procedure, can maintain a spacing between distracted tissues during introduction of an implant and can protect the surrounding tissue from damage caused by inserting implants and instruments into the space between distracted tissues.

Where tissue is distracted for introduction of an instrument and/or implant, introduction of the implant along a conduit requires an internal dimension of the conduit internal channel to be sized to allow passage of the implant, so that the external dimension is greater by twice the conduit wall thickness. This requires temporary over-distraction of the tissue, which is in some cases problematic.

There is therefore a need for a conduit-based implant delivery system which would not require distraction beyond the dimensions of the final implant structure.

### SUMMARY OF THE INVENTION

The present invention is a system and method for deploying an implant within a body.

According to the teachings of an embodiment of the present invention there is provided, a system for deploying a medical implant comprising: (a) a conduit assembly comprising a first conduit portion having an inner channel and a second conduit portion having an inner channel, the second conduit portion being rigidly attached to a distal end of the first conduit portion such that the inner channels of the first conduit portion and the second conduit portion are contiguous, the second conduit portion providing: (i) first and second opposing tissue contact surfaces, and (ii) a rigid bridging structure defining a fixed spacing between the first and second opposing tissue contact surfaces; and (b) an implant insert for introduction along the inner channel of the first conduit portion to a deployed position engaging the second conduit portion, wherein the second conduit portion is selectively detachable from the first conduit portion so as to leave the second conduit portion and the implant insert together defining an implant.

According to a further feature of an embodiment of the present invention, there is also provided a reinforcing element sized for insertion along the inner channels of the first conduit portion and the second conduit portion for enhancing mechanical strength of the conduit assembly during introduction of the second conduit portion into a body.

According to a further feature of an embodiment of the present invention, the conduit assembly has an open end, and wherein the reinforcing element is formed with a bullet-nose tip for facilitating introduction of the second conduit portion into a body.

According to a further feature of an embodiment of the present invention, the second conduit portion is implemented as a three-sided conduit.

According to a further feature of an embodiment of the present invention, the implant insert and the second conduit portion are formed with complementary interlocking features for locking the implant insert against longitudinal motion relative to the second conduit portion.

According to a further feature of an embodiment of the present invention, the second conduit portion is open on at least one side, and wherein the implant insert is configured to expand through the open side.

According to a further feature of an embodiment of the present invention, the inner channels of the first conduit portion and the second conduit portion form a continuous channel of substantially constant internal cross-section.

According to a further feature of an embodiment of the present invention, each of the first and second opposing tissue contact surfaces defines a contact plane which is substantially coplanar with a corresponding surface of the first conduit portion.

According to a further feature of an embodiment of the present invention, there is also provided at least one tool configured for insertion via inner channels.

According to a further feature of an embodiment of the present invention, the tool is configured for removal of biological tissue.

According to a further feature of an embodiment of the present invention, the implant is an intervertebral spacer.

According to a further feature of an embodiment of the present invention, the second conduit portion is rigidly attached to the first conduit portion by a mechanical engagement, the system further comprising a release mechanism configured for insertion along the inner channel of the first conduit portion, the release mechanism being selectively operable to release the mechanical engagement.

There is also provided according to the teachings of an embodiment of the present invention, a method for deploying a medical implant between two tissue surfaces, the method comprising the steps of: (a) introducing between the two tissue surfaces at least part of a conduit assembly comprising a first conduit portion having an inner channel and a second conduit portion having an inner channel, the second conduit portion being rigidly attached to a distal end of the first conduit portion such that the inner channels of the first conduit portion and the second conduit portion are contiguous, the second conduit portion providing: (i) first and second opposing tissue contact surfaces, and (ii) a rigid bridging structure defining a fixed spacing between the first and second opposing tissue contact surfaces, the conduit assembly being introduced so that the second conduit portion reaches a desired implant location between the two tissue surfaces; (b) introducing along the inner channel of the first conduit portion an implant insert so as to reach a deployed position engaging the second conduit portion; and (c) detaching the first conduit portion from the second conduit portion and withdrawing the first conduit portion so that the second conduit portion and the implant insert remain deployed together as an implant.

According to a further feature of an embodiment of the present invention, a reinforcing element is inserted along the inner channels of the first conduit portion and the second conduit portion prior to the step of introducing the conduit assembly, thereby enhancing mechanical strength of the conduit assembly during introduction, the reinforcing element being withdrawn from the inner channels prior to introducing the implant insert.

According to a further feature of an embodiment of the present invention, the conduit assembly has an open end, and wherein the reinforcing element is formed with a bullet-nose tip.

According to a further feature of an embodiment of the present invention, the second conduit portion is implemented as a three-sided conduit.

According to a further feature of an embodiment of the present invention, the implant insert and the second conduit portion are formed with complementary interlocking features for locking the implant insert against longitudinal motion relative to the second conduit portion.

According to a further feature of an embodiment of the present invention, the implant insert is actuated to expand through an open side of the second conduit portion.

According to a further feature of an embodiment of the present invention, at least one tool is inserted via inner channels prior to introduction of the implant insert.

According to a further feature of an embodiment of the present invention, the tool is operated to remove biological tissue.

According to a further feature of an embodiment of the present invention, the implant is deployed as an intervertebral spacer.

According to a further feature of an embodiment of the present invention, a release mechanism inserted along the inner channel of the first conduit portion is employed to release mechanical engagement between the second conduit portion and the first conduit portion.

### BRIEF DESCRIPTION OF THE DRAWINGS

The invention is herein described, by way of example only, with reference to the accompanying drawings, wherein:

FIG. 1 is an isometric view of components of a conduit assembly, prior to assembly, according to an embodiment of the present invention;

FIGS. 2A and 2B are isometric views of the conduit assembly of FIG. 1 partially and fully assembled, respectively;

FIGS. 3A and 3B are a side view and an isometric end view, respectively, of a resilient retainer device from the conduit assembly of FIG. 1;

FIG. 4A is an isometric view of a reinforcing element for use with the conduit assembly of FIG. 2B;

FIG. 4B is an isometric view of the reinforcing element of FIG. 4B inserted into the conduit assembly of FIG. 2B;

FIGS. 5A and 5B are isometric views of a release mechanism for use with the conduit assembly of FIG. 2B, the device being shown in an insertion state and a deployed state, respectively;

FIG. 6 is an isometric view illustrating the positioning of the release mechanism within the partially assembled conduit assembly of FIG. 2A, showing the alignment of the device with windows of the conduit;

FIGS. 7A and 7B are enlarged schematic partial cut-away views illustrating the release mechanism of FIG. 5A prior to and during operation of the release mechanism, respectively;

FIG. 8 is a schematic isometric view of an alternative implementation of a conduit assembly according to a further embodiment of the present invention;

FIGS. 9A and 9B are isometric views of a conduit assembly according to the present invention in use with a tissue removal tool, illustrated before and during deflection of the tool, respectively;

FIGS. 10A and 10B are isometric views of a conduit assembly according to the present invention in use with an alternative tissue removal tool, illustrated before and during deflection of the tool, respectively;

FIGS. 11A and 11B illustrate the conduit assembly of FIG. 2B in use with an implant insert and corresponding holder and deployment mechanism, shown prior to and after insertion of the implant insert into the conduit assembly;

FIGS. 11C and 11D are views similar to FIG. 11B illustrating partial and complete deployment of the implant insert;

FIG. 11E illustrates a final implant structure after disconnection of the implant insert holder and part of the conduit assembly from FIG. 11D;

FIGS. 12A and 12B are partial cut-away views of a distal portion of the assemblies of FIGS. 11B and 11D, respectively, cut along a central longitudinal plane;

FIG. 12C is a longitudinal cross-sectional view taken through the implant of FIG. 11E;

FIG. 13A is an isometric view of a further embodiment of the present invention employing a pair of implant inserts opening in opposite directions;

FIG. 13B is an isometric view of the final deployed implant deployed by the embodiment of FIG. 13A;

FIG. 14A is a partially cut-away views taken along a central longitudinal plane in FIG. 13B; and

FIG. 14B is an enlarged view of a distal portion of FIG. 14A.

### DESCRIPTION OF THE PREFERRED EMBODIMENTS

The present invention is a system and method for deploying an implant within a body.

The principles and operation of systems and methods according to the present invention may be better understood with reference to the drawings and the accompanying description.

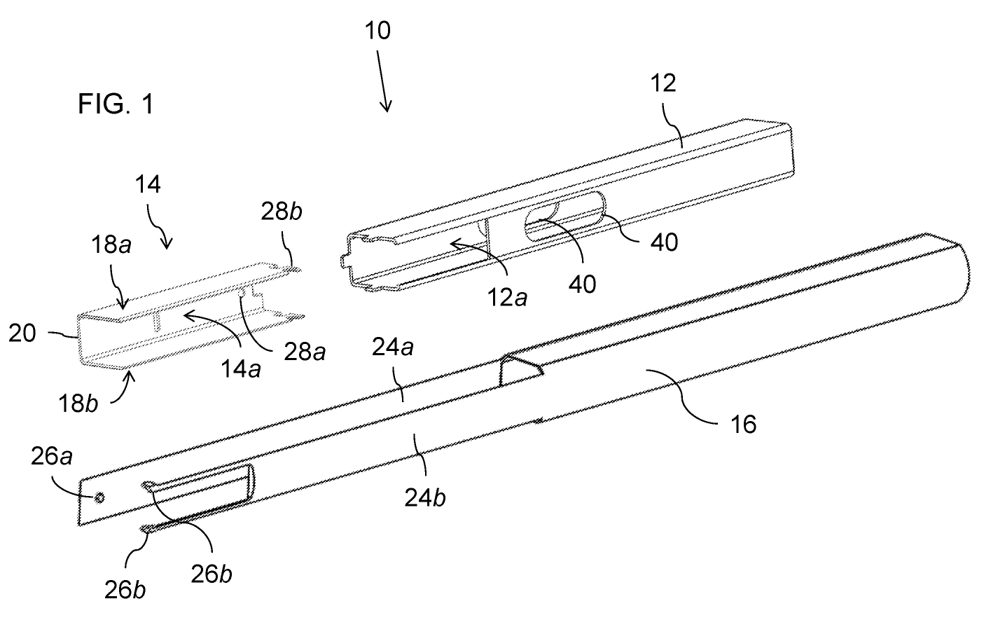

Referring now to the drawings, FIGS. 1-12C illustrate various aspects of a first embodiment of a system and corresponding method constructed and operative for deploying a medical implant. In general terms, the system includes a conduit assembly, generally designated 10, with a first conduit portion 12 and a second conduit portion 14. Second conduit portion 14 is rigidly attached to a distal end of first conduit portion 12, such as by a mechanical engagement arrangement 16 described in more detail below, such that inner channels 12a, 14a of first conduit portion 12 and second conduit portion 14, respectively, are contiguous.

Second conduit portion 14 is configured to provide first and second opposing tissue contact surfaces 18a and 18b which are held in a fixed spaced relation by a rigid bridging structure 20.

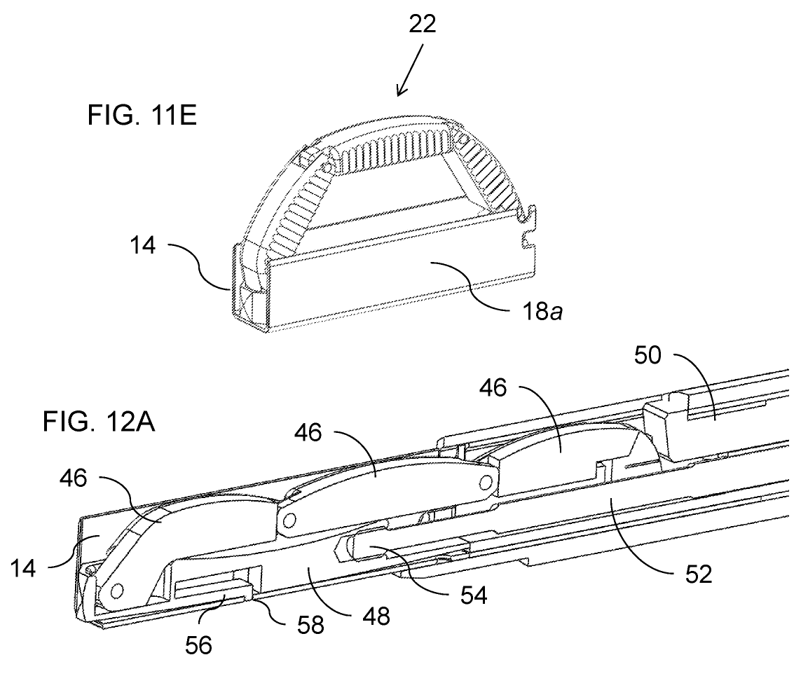

An implant insert 22 (see FIG. 11A) is configured for introduction along inner channel 12a of first conduit portion 12 to a deployed position engaging second conduit portion 14.

Second conduit portion 14 is selectively detachable from first conduit portion 12 so as to leave second conduit portion 14 and implant insert 22 together defining an implant, as illustrated in FIG. 11E.

At this stage, it will already be appreciated that the present invention offers profound advantages. Specifically, conduit assembly 10 provides the functionality of a conduit, with various associated advantages as will be discussed below. However, at the end of the procedure, second conduit portion 14 is detached from the conduit to remain as part of the implant, thereby avoiding over-distraction of the surrounding tissue. This and other advantages of the present invention will be better understood with reference to the drawings and the subsequent description.

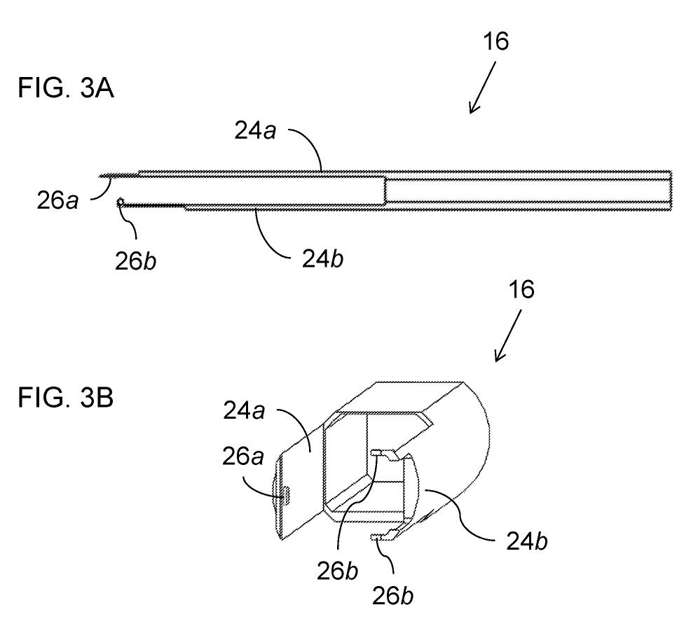

Turning now to the features of the present invention in more detail, second conduit portion 14 is rigidly mounted relative to first conduit portion 12 in a manner which allows detachment when required. In the example illustrated here, the attachment is achieved using mechanical engagement arrangement 16 which features elongated spring elements 24a, 24b with engagement features 26a, 26b deployed for engaging complementary engagement features 28a, 28b formed in second conduit portion 14. Spring elements 24a, 24b are here implemented as elongated leaf springs. Engagement feature 26a is here shown as a cylindrical projection engaging a corresponding round aperture (engagement feature 28a) of second conduit portion 14. Engagement feature 26b is here implemented as a pair of shaped projections which engage corresponding recesses 28b formed in the walls of second conduit portion 14. The features of engagement arrangement 16 are additionally seen in FIGS. 3A and 3B.

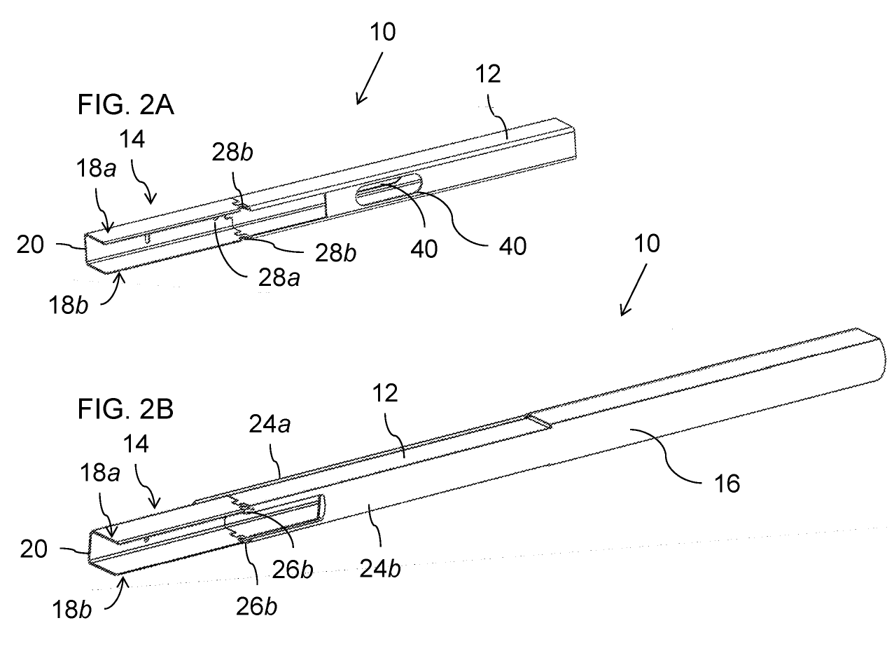

As a result of these engagement features, conduit assembly 10 as illustrated in FIG. 2B functions as a continuous conduit with considerable structural strength, allowing it to be manipulated in a manner similar to a conventional conduit of unitary construction. Additionally, in order to provide functionality similar to a conventional conduit, inner channels 12a and 14a preferably form a continuous channel of constant cross-section (neglecting the various apertures, slots and open sides such as are described elsewhere herein), so as to allow a close-fitting insert, tool or other device to slide smoothly along the conduit.

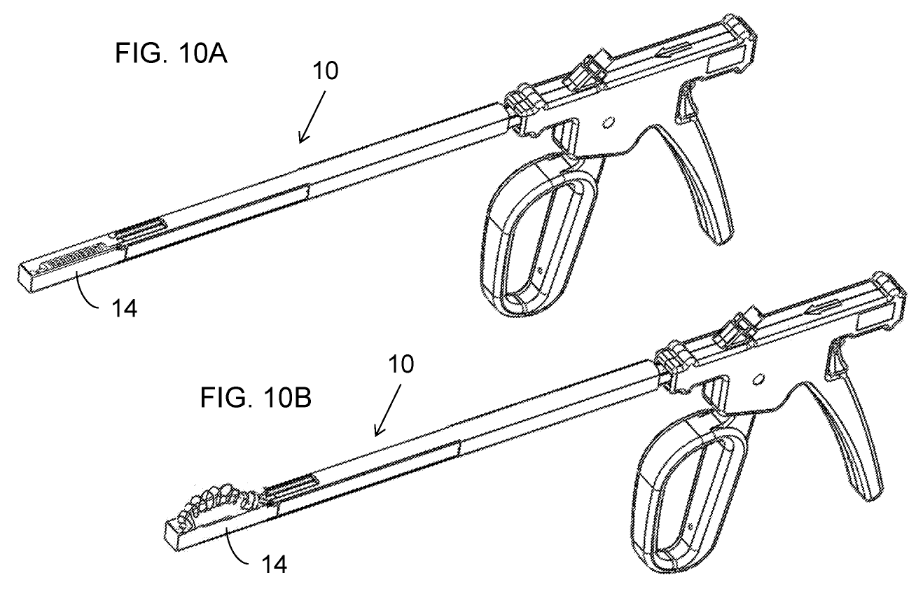

Although illustrated here as part of a separate engagement arrangement 16, it should be noted that the various spring elements 24a, 24b and engagement features 26a, 26b can alternatively be directly attached to, or otherwise integrated into, first conduit portion 12. An implementation of this type is illustrated schematically in FIG. 8. Additionally, or alternatively, instead of resilient springs, elements 24a and 24b may be implemented as deformable elements which are plastically deformed beyond their elastic limit during release of second conduit portion 14, thereby preventing reuse of a device designed for single use.

Furthermore, although illustrated herein with spring-biased interlocking features, a wide range of other forms of detachable interconnections between second conduit portion 14 and first conduit portion 12 may be used. Examples include, but are not limited to: other forms of spring-biased interlocking mechanisms; various forms of engagement employing locking pins or bolts; and various break-away features and combinations thereof.

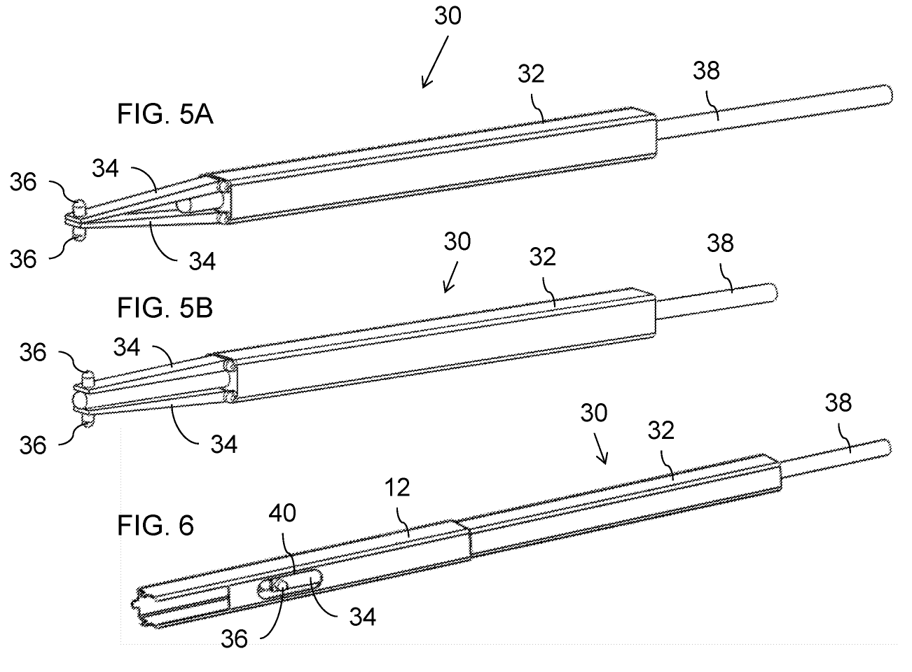

For whichever form of engagement is chosen, the present invention preferably provides an arrangement for selectively releasing engagement of second conduit portion 14. In the particularly preferred but non-limiting example of engagement arrangement 16 as illustrated herein, release of the engagement is achieved by applying outward pressure to spring elements 24a, 24b which displaces engagement features 26a, 26b outwards to release engagement from recesses 28a, 28b. One non-limiting example of a release mechanism 30 for this purpose is illustrated in FIGS. 5A-7B.

Referring specifically to FIGS. 5A and 5B, release mechanism 30 as shown here includes a body 32 with two hinged flaps 34 each provided with an outwardly projecting pin 36. In an initial state as shown in FIG. 5A, the flaps are folded together so that pins 36 lie within the rectangular profile of body 32. When an actuator rod 38 is advanced, it pushes flaps 34 apart so as to urge pins 36 outwards.

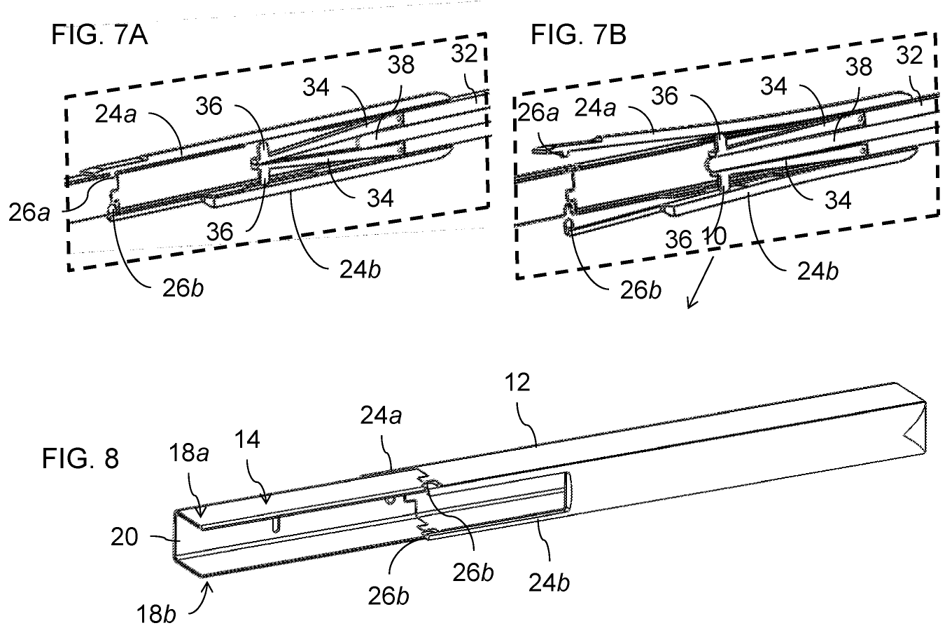

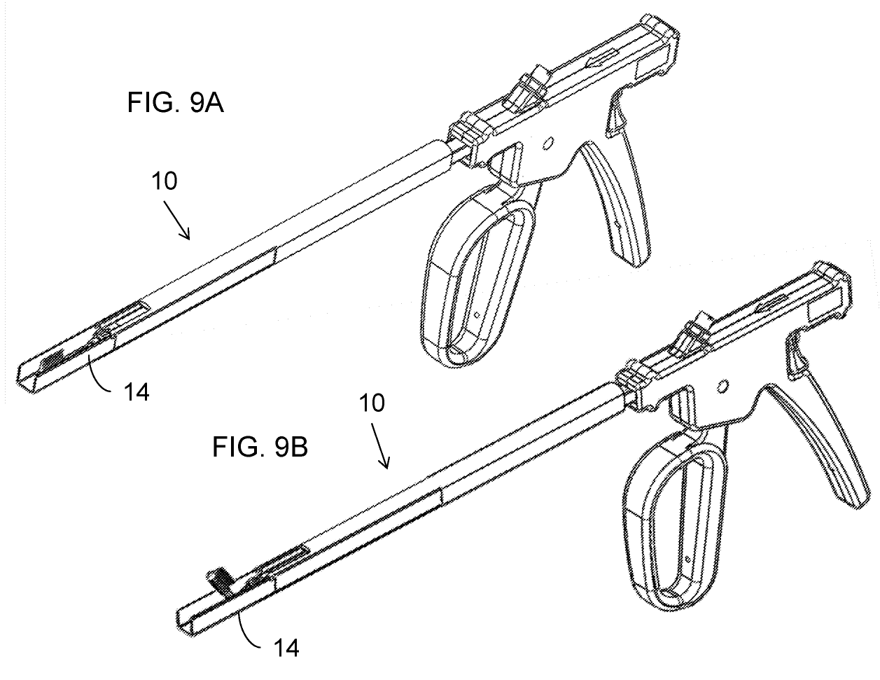

FIG. 6 illustrates the preferred deployed position of release mechanism 30 within first conduit portion 12, with pins 36 opposite bilateral windows 40 cut out of the side walls of the first conduit portion 12. This positioning allows pins 36 to bear directly on the inside surfaces of spring elements 24a, 24b, thereby pushing the spring elements outwards and releasing engagement of second conduit portion. This release process is illustrated in FIGS. 7A and 7B.

It should be noted that any increase in the dimensions of conduit assembly 10 due to engagement arrangement 16, and any expansion of dimensions of the assembly during release of the engagement, preferably occur in a direction perpendicular to a direction of distraction of tissue. Specifically, a direction of tissue distraction is typically roughly perpendicular to tissue contact surfaces 18a and 18b, referred to herein by way of a non-limiting exemplary terminology as an “axial” or “vertical” direction, whereas the increased wall thickness of conduit assembly 10 due to engagement arrangement 16, and the opening motion of spring elements 24a and 24b are both “in-plane” or “lateral”, i.e., in a plane parallel to the planes of tissue contact surfaces 18a and 18b, and typically within a “slice” bounded by those two planes. It should also be noted that the opening motion illustrated in FIG. 7B is exaggerated for clarity of presentation, and that a much smaller motion is sufficient to effect release of second conduit portion 14.

Furthermore, according to certain particularly preferred implementations of the present invention, first and second opposing tissue contact surfaces 18a and 18b define contact planes which are substantially coplanar with corresponding surfaces of first conduit portion 12. It will be noted that tissue contact surfaces 18a and 18b are in certain embodiments implemented with various projecting ridges or the like (not shown) for enhanced bone purchasing and migration resistance. In such cases, the “contact plane” is taken to be the plane of the underlying surface when ignoring the projections.

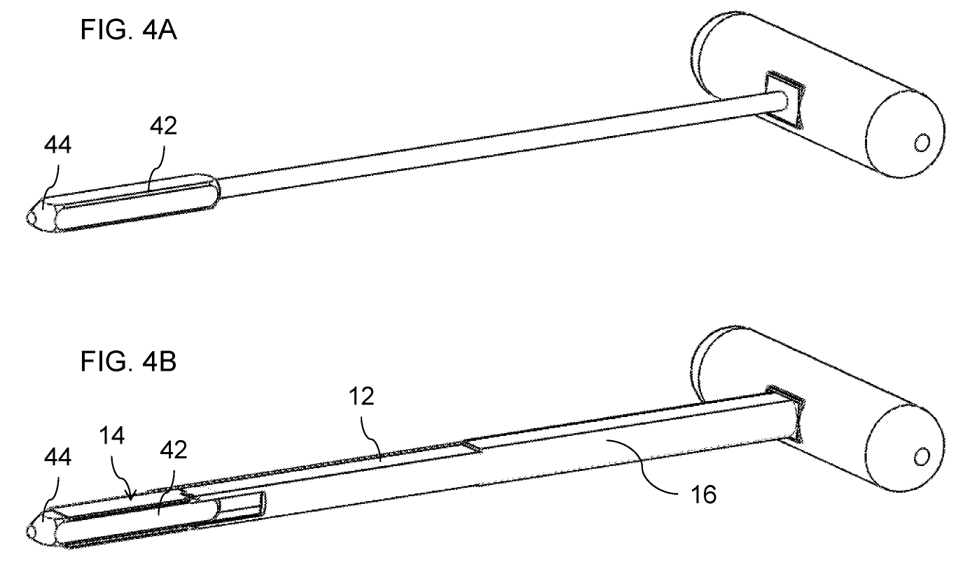

Turning now to FIGS. 4A and 4B, these illustrate a further optional but preferred feature of certain embodiments of the present invention. Specifically, there is shown in FIG. 4A a reinforcing element 42 sized for insertion along inner channels 12a and 14a of first conduit portion 12 and second conduit portion 14, for enhancing mechanical strength of conduit assembly 10 during introduction into a body. The operative portion of reinforcing element 42 is preferably a solid block of cross-sectional size and shape to fit closely within the inner channels as shown in FIG. 4B, thereby lending rigidity and strength to the structure. This option is particularly valuable in certain orthopedic applications in which large forces and/or hammer impacts may be used to achieve correct positioning of the device between regions of hard tissue.

Most preferably, reinforcing element 42 is formed with a “bullet-nose tip” 44, i.e., a tip which is tapered in one or two dimensions, and has a rounded tip. The bullet-nose tip facilitates insertion of the device while minimizing damage or trauma to surrounding tissue. According to this option, conduit assembly 10 is implemented with an open end, and reinforcing element 42 is configured to reach a fully-inserted position where the bullet-nose tip 44 protrudes from the open end.

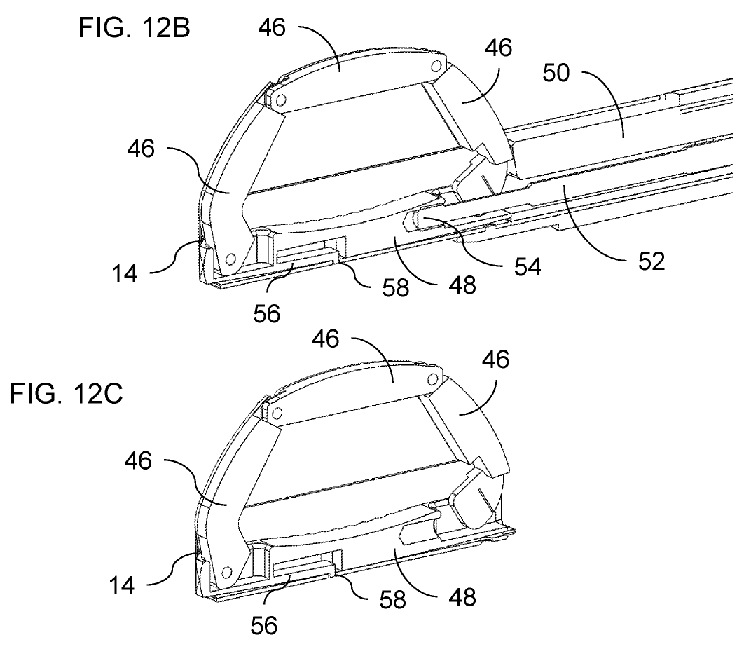

As mentioned above, a final implant according to the present invention is formed by a combination of second conduit portion 14 together with at least one subsequently introduced implant insert, exemplified here by implant insert 22 as shown in FIGS. 11A-12C. In this particularly preferred but non-limiting example, second conduit portion 14 is implemented as a three-sided conduit, and implant insert 22 is configured to expand through the open side, thereby increasing the in-plane footprint of the final implant. By way of one non-limiting example of an expanding structure, as best seen in FIGS. 12A-12C, implant insert 22 is illustrated here as a series of hingedly interconnected segments 46 hingedly anchored at a distal end to a base 48. A pusher rod 50 displaces a proximal end of the sequence of segments 46 so as to deflect the segments into an arched form, as shown in FIG. 12B, where the arched form may be locked by any suitable locking mechanism (not shown).

Insertion of implant insert 22 is preferably controlled by a manually advanced holder 52 implemented here as a rod with a threaded tip 54 which releasably engages base 48. Base 48 is preferably implemented with a resilient locking element 56 deployed to engage a complementary slot 58 in second conduit portion 14, thereby interlocking implant insert 22 and second conduit portion 14 in a desired spatial relation and preventing subsequent longitudinal and/or lateral motion of implant insert 22 relative to second conduit portion 14.

It will be noted that implant insert 22, particularly when including an expansion mechanism, may be considerably less robust than the structure of conduit assembly 10. The fact that implant insert 22 is inserted only after correct positioning of distal conduit component 14 has been achieved ensures that implant insert 22 is not exposed to hammer blows or any other high-stress conditions which may be used during the insertion process, thereby helping to protect implant insert 22 from damage.

Clearly, details of the structure of implant insert 22 are chosen according to the intended application. The non-limiting example illustrated here is believed to be of particular value as an intervertebral spacer introduced as part of an intervertebral fusion procedure.

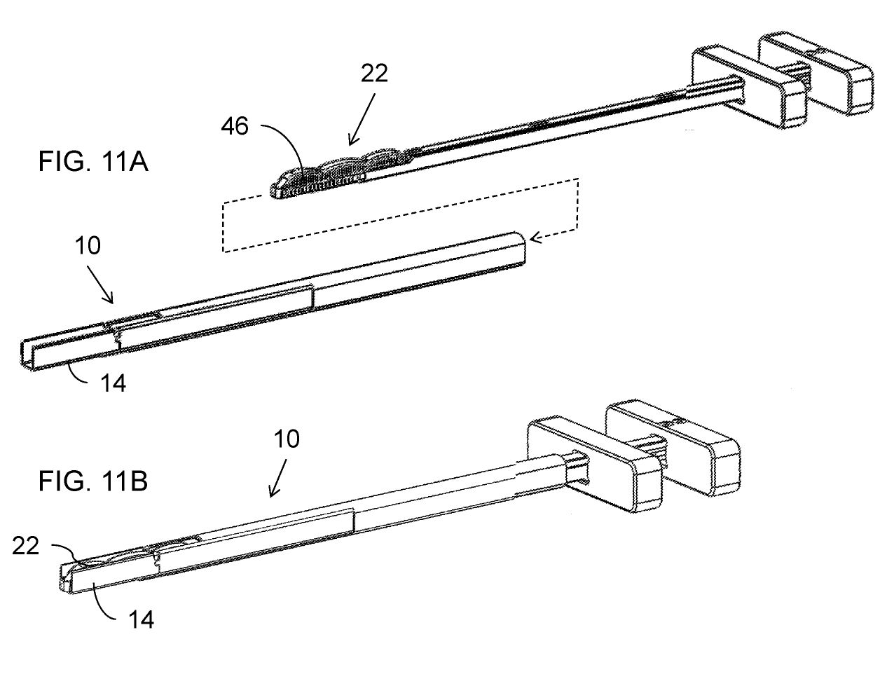

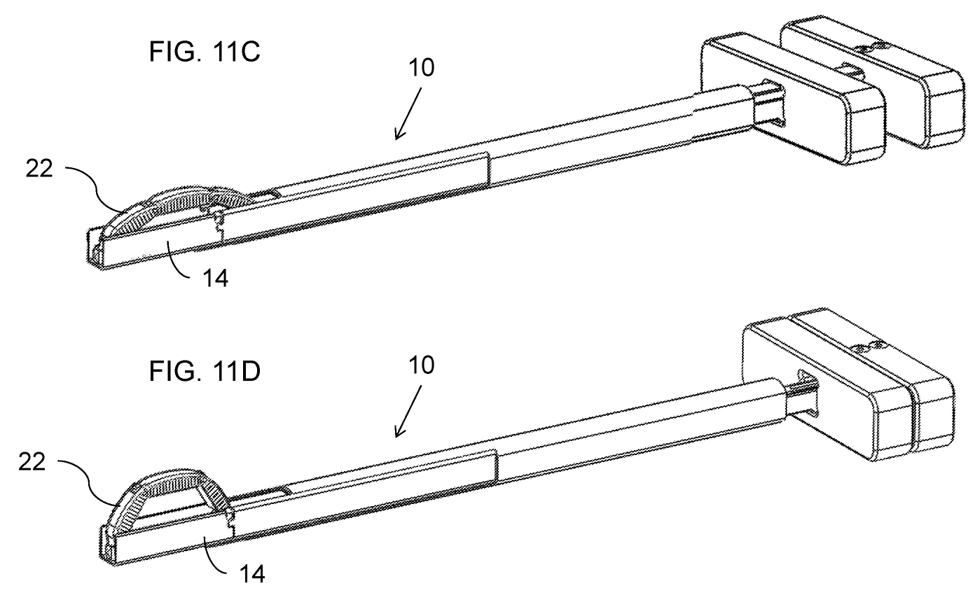

According to a further particularly preferred feature of certain implementations of the present invention, conduit assembly 10 can be used to advantage for introduction of at least one tool via inner channels 12a and 14a for performing one or more function at or near the target implant location. By way of one particularly preferred but non-limiting example, the tool may be configured for removal of biological tissue in order to make space for introduction and/or expansion of implant insert 22. In the context of an intervertebral implant, the tool may thus be a discectomy tool for removing part or all of the tissue of a damaged intervertebral disc. By way of non-limiting examples, FIGS. 9A-9B illustrate implementation of a system according to an embodiment of the present invention employing a discectomy tool according to the teachings of co-assigned PCT Patent Application Publication No. WO 2012/153319 A1, while FIGS. 10A-10B illustrate implementation of a system according to an embodiment of the present invention employing a discectomy tool according to the teachings of co-assigned PCT Patent Application Publication No. WO 2013/171664 A1.

At this stage, the operation of a system according to the present invention, corresponding also to a method according to the present invention, will be clearly understood, as follows:
Optionally, where needed, a preparatory drilling and/or distraction process may be used to facilitate introduction of the device.
Conduit assembly 10 is then introduced between two tissue surfaces, optionally with reinforcing element 42 inserted, until second conduit portion 14 reaches a desired implant location between the two tissue surfaces.
If used, reinforcing element 42 is then removed to free up the inner channel of conduit assembly 10.
If desired, one or more tool, such as a discectomy tool, is inserted through conduit assembly 10 to perform one or more surgical procedures.
Implant insert 22 is then introduced along the inner channel of first conduit portion 12 so as to reach a deployed position engaging second conduit portion 14.
If relevant, implant insert 22 is deployed, such as to an expanded state.
Optionally, fusion promoting material (such as auto/allo bone graft or BMP or similar) may be inserted through the first conduit portion 12 into the expanded implant.
First conduit portion 12 is then detached from second conduit portion 14 and first conduit portion 12 is withdrawn so that second conduit portion 14 and implant insert 22 remain deployed together as an implant.

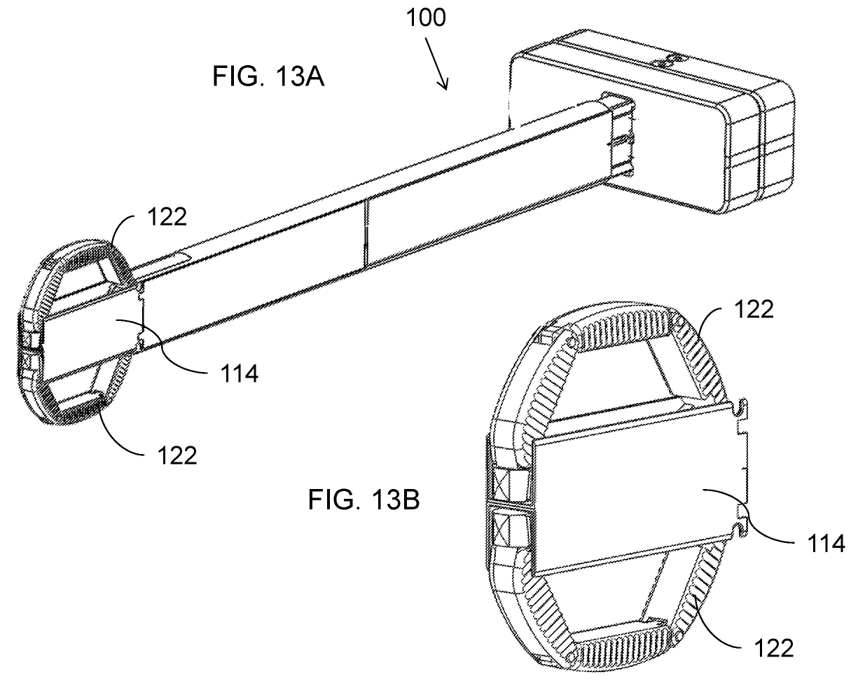

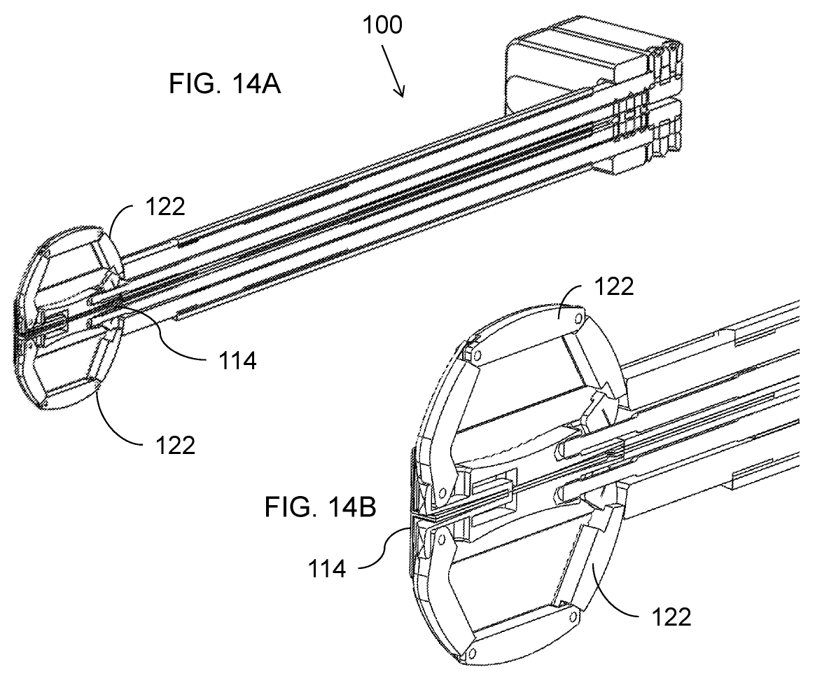

Turning finally to FIGS. 13A-14B, there is shown a further embodiment of the present invention, generally designated 100. This embodiment is structurally and functionally similar to the embodiment described above, but employs a doubled structure in which a second conduit portion 114 is implemented as an I-beam equivalent to two three-sided conduits deployed back-to-back, and each side of second conduit portion 114 receives a corresponding implant insert 122. This results in a bilaterally expanded structure. In all other respects, the structure and function of system 100 may be understood by analogy to the corresponding description of the above embodiments. The implant components may be inserted and/or expanded simultaneously or independently.

It will be appreciated that the above descriptions are intended only to serve as examples, and that many other embodiments are possible within the scope of the present invention as defined in the appended claims.

What is claimed is:

1. A system for deploying a medical implant comprising:
(a) a conduit assembly comprising a first conduit portion having an inner channel and a second conduit portion having an inner channel, said second conduit portion being rigidly attached to a distal end of said first conduit portion such that said inner channels of said first conduit portion and said second conduit portion are contiguous, said second conduit portion providing:
(i) first and second opposing tissue contact surfaces, and
(ii) a rigid bridging structure defining a fixed spacing between said first and second opposing tissue contact surfaces; and
(b) an implant insert for introduction along said inner channel of said first conduit portion to a deployed position engaging said second conduit portion,
wherein said second conduit portion is selectively detachable from said first conduit portion so as to leave said second conduit portion and said implant insert together defining an implant.

2. The system of claim 1, further comprising a reinforcing element sized for insertion along said inner channels of said first conduit portion and said second conduit portion for enhancing mechanical strength of said conduit assembly during introduction of said second conduit portion into a body.

3. The system of claim 2, wherein said conduit assembly has an open end, and wherein said reinforcing element is formed with a bullet-nose tip for facilitating introduction of said second conduit portion into a body.

4. The system of claim 1, wherein said second conduit portion is implemented as a three-sided conduit.

5. The system of claim 1, wherein said implant insert and said second conduit portion are formed with complementary interlocking features for locking said implant insert against longitudinal motion relative to said second conduit portion.

6. The system of claim 1, wherein said second conduit portion is open on at least one side, and wherein said implant insert is configured to expand through said open side.

7. The system of claim 1, wherein said inner channels of said first conduit portion and said second conduit portion form a continuous channel of substantially constant internal cross-section.

8. The system of claim 1, wherein each of said first and second opposing tissue contact surfaces defines a contact plane which is substantially coplanar with a corresponding surface of said first conduit portion.

9. The system of claim 1, further comprising at least one tool configured for insertion via inner channels.

10. The system of claim 9, wherein said tool is configured for removal of biological tissue.

11. The system of claim 1, wherein said implant is an intervertebral spacer.

12. The system of claim 1, wherein said second conduit portion is rigidly attached to said first conduit portion by a mechanical engagement, the system further comprising a release mechanism configured for insertion along said inner channel of said first conduit portion, said release mechanism being selectively operable to release said mechanical engagement.

13. A method for deploying a medical implant between two tissue surfaces, the method comprising the steps of:
(a) introducing between the two tissue surfaces at least part of a conduit assembly comprising a first conduit portion having an inner channel and a second conduit portion having an inner channel, said second conduit portion being rigidly attached to a distal end of said first conduit portion such that said inner channels of said first conduit portion and said second conduit portion are contiguous, said second conduit portion providing:
(i) first and second opposing tissue contact surfaces, and
(ii) a rigid bridging structure defining a fixed spacing between said first and second opposing tissue contact surfaces,
said conduit assembly being introduced so that said second conduit portion reaches a desired implant location between the two tissue surfaces;
(b) introducing along said inner channel of said first conduit portion an implant insert so as to reach a deployed position engaging said second conduit portion; and
(c) detaching said first conduit portion from said second conduit portion and withdrawing said first conduit portion so that said second conduit portion and said implant insert remain deployed together as an implant.

14. The method of claim 13, further comprising inserting a reinforcing element along said inner channels of said first conduit portion and said second conduit portion prior to said step of introducing said conduit assembly, thereby enhancing mechanical strength of said conduit assembly during introduction, said reinforcing element being withdrawn from said inner channels prior to introducing the implant insert.

15. The method of claim 14, wherein said conduit assembly has an open end, and wherein said reinforcing element is formed with a bullet-nose tip.

16. The method of claim 13, wherein said second conduit portion is implemented as a three-sided conduit.

17. The method of claim 13, wherein said implant insert and said second conduit portion are formed with complementary interlocking features for locking said implant insert against longitudinal motion relative to said second conduit portion.

18. The method of claim 13, further comprising actuating said implant insert to expand through an open side of said second conduit portion.

19. The method of claim 13, further comprising inserting at least one tool via inner channels prior to introduction of said implant insert.

20. The method of claim 19, wherein said tool is operated to remove biological tissue.

21. The method of claim 13, wherein said implant is deployed as an intervertebral spacer.

22. The method of claim 13, further comprising employing a release mechanism inserted along said inner channel of said first conduit portion to release mechanical engagement between said second conduit portion and said first conduit portion.
